SEQUENCE LISTING (7 sequences)

SEQ ID NO: 1 - DNA, 5789 bp, Arabidopsis thaliana
gtttcttcttcatggagacttgaaagcttttaaagtatatctaaaaacgcagtcgttttaagactgtgtgtgagaaatggctctgtttagagatattgttcttcttgggtttctcttctgcttgagcttagtagctactgtgacttcagaggagggtcagttattatactgatgcatgcttcttcaagttcaagattttcgtctttttgttttatattagtgaaaaaaacttaaagatgagatttttatatgatttttgaagtttcatttggtgaaaatgagatctgggtacttgttattttctatttttgctttttgtaatggtttttttttacttggtgggtcttctatagaatcaaaagaagctttgaataaattagggtttgagttttattttgttttcttggaagttgaatttttaatcttctcaagaactgacaaatatttttttttgtttttgtgcgtgtgtgttaataaaatatccttaaaacaaaattaaaggagcaacgttgctggagattaagaagtcattcaaagatgtgaacaatgttctttatgactggacaacttcaccttcttcggattattgtgtctggagaggtgtgtcttgtgaaaatgtcaccttcaatgttgttgctctgtaagtttcttcattcctttagattactattacagtggtttttggtgttcttgtgggaaaaagttgtaatttgttttgtgtgtgttttctatgttttgtagtaatttgtcagatttgaatcttgatggagaaatctcacctgctattggagatctcaagagtctcttgtcaatgtaactgtttcaacattcactgtagcatgaaataaagtatcttactttaattctattccactctctgagttgtgacttttgtcttctgtttttttctaatgtagtgatctgcgaggtaatcgcttgtctggacaaatccctgatgagattggtgactgttcttctttgcaaaacttgtaagaacagtgattggtgttattctaccattaaacttttgttcatagaggttttatttgatgaagtgtgttcatgttgttttttattcagagacttatccttcaatgaattaagtggtgacataccgttttcgatttcgaagttgaagcaacttgagcagctgtaagtagctagttattctgctactagtcttcatatgtcattgctaaaaatatactcaccatgtggaatatggatttttactttgtccaggattctgaagaataaccaattgataggaccgatcccttcaacactttcacagattccaaacctgaaaattctgtatgttccccatgattcttacatgtcttactacttttagctatataggtgatcatacatgtgtaatttcaattgcagggacttggcacagaataaactcagtggtgagataccaagacttatttactggaatgaagttcttcagtatctgtaagtgtcaatgttttttgaagtctgtcaatgtctcttcattacccggtgataattgttgtactatgatgagcagtgggttgcgaggaaacaacttagtcggtaacatttctccagatttgtgtcaactgactggtctttggtatttgtgagtcttcttgcacatctgaatagtatgatgagttcttttgtaaatatcaaatatctgactttgttttgatattgaatcagtgacgtaagaaacaacagtttgactggtagtatacctgagacgataggaaattgcactgccttccaggttttgtatgtgcctctttctctacttctaaacatcattactgtaatttgggttacttaagaaaatctacttaactggtttgcttattacgaactcagggacttgtcctacaatcagctaactggtgagatcccttttgacatcggcttcctgcaagttgcaacattgttagttctcacctctactaatcttttgctttaaattttggctagcctttgttttcttttaaagaagatcattttcttatcttagatcattgcaaggcaatcaactctctgggaagattccatcagtgattggtctcatgcaagcccttgcagtcttgtaagtacttttcttctaatcaatgaagctacttataacattttcatgaacttaggttatatgttttcttttacagagatctaagtggcaacttgttgagtggatctattcctccgattctcggaaatcttactttcaccgagaaattgtaattctttacctgtttgttttcagtttggagtcaaatgtcataccatgttaatgatagtgatttatctttttggctttatctctaggtatttgcacagtaacaagctgactggttcaattccacctgagcttggaaacatgtcaaaactccattacctgtatgaccaaccttctcttcacttctctttttgcatacagtcactactaagttgtgtttccttatcaactatttgtaaaatattcatagggaactcaatgataatcatctcacgggtcatataccaccagagcttgggaagcttactgacttgtttgatctgtaagtagttcttcctatgcttgacatgttttgatgttcttatgcttatatgaactatgtacatataggaatgtggccaacaatgatctggaaggacctatacctgatcatctgagctcttgcacaaatctaaacagcttgtatgtatctctttctctgaaaacttctcacttgaatgttcaagattggtgctttatatgattttgtgtctcattaatgtaatgtagaaatgttcatgggaacaagtttagtggcactataccccgagcatttcaaaagctagaaagtatgacttacctgtaagtatcgacgctgagaatttctctaatcttatataatatatagttccacagcgtttgttttttcgaatttcaagtcattaactactgagtttttggttgcctttgatttatcggttcaaccagtaatctgtccagcaacaatatcaaaggtccaatcccggttgagctatctcgtatcggtaacttagatacattgtaagtgtttcttgttttctgtgaagtatacatcattatatgtgccttgtctcacatttattaaatttaatgacatttgaagggatctttccaacaacaagataaatggaatcattccttcttcccttggtgatttggagcatcttctcaagatgtgagcatccataagacctccagttttattgtttatttctagcaaaagatgaaaatggtttgtgaactcttgcattcttgttataggaacttgagtagaaatcatataactggtgtagttccaggcgactttggaaatctaagaagcatcatggaaatgtaagaagttaacttctatctgcttggttagagtttttttcatttatctcaattactgttctgaatttgtgtgtttgtggttgcagagatctttcaaataatgatatctctggcccaattccagaagagcttaaccaattacagaacataattttgctgtaagcaatcttcctcttatcccttccaagctgttaagaaattgtttttgtagaatgaaactaaaactctgtatacacaataatgaggtcactatagtgtgatccaggaacatgtattgggttggtgatctatctaatgttgtgtttcttaaaattgcttgcaggagactggaaaataataacctgactggtaatgttggttcattagccaactgtctcagtctcactgtattgtaagtaggcacctttggttctgaaacattttttgtccctctttgtgcatcttttgctaagaatataaccctgcaatcttcactaactcttataggaatgtatctcataacaacctcgtaggtgatatccctaagaacaataacttctcaagattttcaccagacaggtatggtaatttagcaggttttggtattgtgcattttgttttgtttgctaatatctatgtttatgtttttggataaagcttcattggcaatcctggtctttgcggtagttggctaaactcaccgtgtcatgattctcgtcgaactgtacgaggtgattacattcttctaaaagcttccattcacaaaacctaagataattaaagctcatgtttctatccatgttttgtctgcagtgtcaatctctagagcagctattcttggaatagctattgggggacttgtgatccttctcatggtcttaatagcagcttgccgaccgcataatcctcctccttttcttgatggatcacttgacaaaccaggtctactctccaaaccactttacgaatgttcttcacctacaatgtaatccaatagttaatccttaaatttcctggtgacatcagtaacttattcgacaccgaagctcgtcatccttcatatgaacatggcactccacgtttacgaggatatcatgagaatgacagagaatctaagtgagaagtatatcattgggcacggagcatcaagcactgtatacaaatgtgttttgaagaattgtaaaccggttgcgattaagcggctttactctcacaacccacagtcaatgaaacagtttgaaacagaactcgagatgctaagtagcatcaagcacagaaatcttgtgagcctacaagcttattccctctctcacttggggagtcttctgttctatgactatttggaaaatggtagcctctgggatcttcttcatggtaagtctcatcgccaaacatagaaaattatttgaatcttctgtgacataacaacttgcttgtgtgttttgtaaaggccctacgaagaaaaagactcttgattgggacacacggcttaagatagcatatggtgcagcacaaggtttagcttatctacaccatgactgtagtccaaggatcattcacagagacgtgaagtcgtccaacattctcttggacaaagacttagaggctcgtttgacagattttggaatagcgaaaagcttgtgtgtgtcaaagtcacatacttcaacttacgtgatgggcacgataggttacatagaccccgagtatgctcgcacttcacggctcactgagaaatccgatgtctacagttatggaatagtccttcttgagttgttaacccgaaggaaagccgttgatgacgaatccaatctccaccatctggtttgttctttcttgcctatctctctcagctgctctgtttaggtcaagtccgtaatcttgttttcattgattcacttacatcagataatgtcaaagacggggaacaatgaagtgatggaaatggcagatccagacatcacatcgacgtgtaaagatctcggtgtggtgaagaaagttttccaactggcactcctatgcaccaaaagacagccgaatgatcgacccacaatgcaccaggtgactcgtgttctcggcagttttatgctatcggaacaaccacctgctgcgactgacacgtcagcgacgctggctggttcgtgctacgtcgatgagtatgcaaatctcaagactcctcattctgtcaattgctcttccatgagtgcttctgatgctcaactgtttcttcggtttggacaagttatttctcagaacagtgagtagtttttcgttaggaggagaatctttaaaacggtatcttttcgttgcgttaagctgttagaaaaattaatgtctcatgtaaagtattatgcactgccttattattattagacaagtgtgtggtgtgaatatgtcttcagactggcacttagacttcctataagttcttgcctatctaagtttttct

SEQ ID NO: 2 - DNA, 2931 bp, Arabidopsis thaliana
atggctctgtttagagatattgttcttcttgggtttctcttctgcttgagcttagtagctactgtgacttcagaggagggagcaacgttgctggagattaagaagtcattcaaagatgtgaacaatgttctttatgactggacaacttcaccttcttcggattattgtgtctggagaggtgtgtcttgtgaaaatgtcaccttcaatgttgttgctcttaatttgtcagatttgaatcttgatggagaaatctcacctgctattggagatctcaagagtctcttgtcaattgatctgcgaggtaatcgcttgtctggacaaatccctgatgagattggtgactgttcttctttgcaaaacttagacttatccttcaatgaattaagtggtgacataccgttttcgatttcgaagttgaagcaacttgagcagctgattctgaagaataaccaattgataggaccgatcccttcaacactttcacagattccaaacctgaaaattctggacttggcacagaataaactcagtggtgagataccaagacttatttactggaatgaagttcttcagtatcttgggttgcgaggaaacaacttagtcggtaacatttctccagatttgtgtcaactgactggtctttggtattttgacgtaagaaacaacagtttgactggtagtatacctgagacgataggaaattgcactgccttccaggttttggacttgtcctacaatcagctaactggtgagatcccttttgacatcggcttcctgcaagttgcaacattatcattgcaaggcaatcaactctctgggaagattccatcagtgattggtctcatgcaagcccttgcagtcttagatctaagtggcaacttgttgagtggatctattcctccgattctcggaaatcttactttcaccgagaaattgtatttgcacagtaacaagctgactggttcaattccacctgagcttggaaacatgtcaaaactccattacctggaactcaatgataatcatctcacgggtcatataccaccagagcttgggaagcttactgacttgtttgatctgaatgtggccaacaatgatctggaaggacctatacctgatcatctgagctcttgcacaaatctaaacagcttaaatgttcatgggaacaagtttagtggcactataccccgagcatttcaaaagctagaaagtatgacttaccttaatctgtccagcaacaatatcaaaggtccaatcccggttgagctatctcgtatcggtaacttagatacattggatctttccaacaacaagataaatggaatcattccttcttcccttggtgatttggagcatcttctcaagatgaacttgagtagaaatcatataactggtgtagttccaggcgactttggaaatctaagaagcatcatggaaatagatctttcaaataatgatatctctggcccaattccagaagagcttaaccaattacagaacataattttgctgagactggaaaataataacctgactggtaatgttggttcattagccaactgtctcagtctcactgtattgaatgtatctcataacaacctcgtaggtgatatccctaagaacaataacttctcaagattttcaccagacagcttcattggcaatcctggtctttgcggtagttggctaaactcaccgtgtcatgattctcgtcgaactgtacgagtgtcaatctctagagcagctattcttggaatagctattgggggacttgtgatccttctcatggtcttaatagcagcttgccgaccgcataatcctcctccttttcttgatggatcacttgacaaaccagtaacttattcgacaccgaagctcgtcatccttcatatgaacatggcactccacgtttacgaggatatcatgagaatgacagagaatctaagtgagaagtatatcattgggcacggagcatcaagcactgtatacaaatgtgttttgaagaattgtaaaccggttgcgattaagcggctttactctcacaacccacagtcaatgaaacagtttgaaacagaactcgagatgctaagtagcatcaagcacagaaatcttgtgagcctacaagcttattccctctctcacttggggagtcttctgttctatgactatttggaaaatggtagcctctgggatcttcttcatggccctacgaagaaaaagactcttgattgggacacacggcttaagatagcatatggtgcagcacaaggtttagcttatctacaccatgactgtagtccaaggatcattcacagagacgtgaagtcgtccaacattctcttggacaaagacttagaggctcgtttgacagattttggaatagcgaaaagcttgtgtgtgtcaaagtcacatacttcaacttacgtgatgggcacgataggttacatagaccccgagtatgctcgcacttcacggctcactgagaaatccgatgtctacagttatggaatagtccttcttgagctgttaacccgaaggaaagccgttgatgacgaatccaatctccaccatctgataatgtcaaagacggggaacaatgaagtgatggaaatggcagatccagacatcacatcgacgtgtaaagatctcggtgtggtgaagaaagttttccaactggcactcctatgcaccaaaagacagccgaatgatcgacccacaatgcaccaggtgactcgtgttctcggcagttttatgctatcggaacaaccacctgctgcgactgacacgtcagcgacgctggctggttcgtgctacgtcgatgagtatgcaaatctcaagactcctcattctgtcaattgctcttccatgagtgcttctgatgctcaactgtttcttcggtttggacaagttatttctcagaacagtgagtag

SEQ ID NO: 3 - protein, 976 aa, Arabidopsis thaliana
MALFRDIVLLGFLFCLSLVATVTSEEGATLLEIKKSFKDVNNVLYDWTTSPSSDYCVWRGVSCENVTFNVVALNLSDLNLDGEISPAIGDLKSLLSIDLRGNRLSGQIPDEIGDCSSLQNLDLSFNELSGDIPFSISKLKQLEQLILKNNQLIGPIPSTLSQIPNLKILDLAQNKLSGEIPRLIYWNEVLQYLGLRGNNLVGNISPDLCQLTGLWYFDVRNNSLTGSIPETIGNCTAFQVLDLSYNQLTGEIPFDIGFLQVATLSLQGNQLSGKIPSVIGLMQALAVLDLSGNLLSGSIPPILGNLTFTEKLYLHSNKLTGSIPPELGNMSKLHYLELNDNHLTGHIPPELGKLTDLFDLNVANNDLEGPIPDHLSSCTNLNSLNVHGNKFSGTIPRAFQKLESMTYLNLSSNNIKGPIPVELSRIGNLDTLDLSNNKINGIIPSSLGDLEHLLKMNLSRNHITGVVPGDFGNLRSIMEIDLSNNDISGPIPEELNQLQNIILLRLENNNLTGNVGSLANCLSLTVLNVSHNNLVGDIPKNNNFSRFSPDSFIGNPGLCGSWLNSPCHDSRRTVRVSISRAAILGIAIGGLVILLMVLIAACRPHNPPPFLDGSLDKPVTYSTPKLVILHMNMALHVYEDIMRMTENLSEKYIIGHGASSTVYKCVLKNCKPVAIKRLYSHNPQSMKQFETELEMLSSIKHRNLVSLQAYSLSHLGSLLFYDYLENGSLWDLLHGPTKKKTLDWDTRLKIAYGAAQGLAYLHHDCSPRIIHRDVKSSNILLDKDLEARLTDFGIAKSLCVSKSHTSTYVMGTIGYIDPEYARTSRLTEKSDVYSYGIVLLELLTRRKAVDDESNLHHLIMSKTGNNEVMEMADPDITSTCKDLGVVKKVFQLALLCTKRQPNDRPTMHQVTRVLGSFMLSEQPPAATDTSATLAGSCYVDEYANLKTPHSVNCSSMSASDAQLFLRFGQVISQNSE

SEQ ID NO: 4 - DNA, 28 bp, Artificial Sequence
  Primer
tatcgatgtatatctaaaaacgcagtcg

SEQ ID NO: 5 - DNA, 23 bp, Artificial Sequence
  Primer
aatatttgtcagttcttgagaag

SEQ ID NO: 6 - DNA, 19 bp, Artificial Sequence
  Primer
gaactcgccgtaaagactg

SEQ ID NO: 7 - DNA, 23 bp, Artificial Sequence
  Primer
tgacttcttaatctccagcaacg

(19) United States
(12) Patent Application Publication
(10) Pub. No.: US 2014/0317782 A1
(43) Pub. Date: Oct. 23, 2014

(54) HIGH TEMPERATURE RESISTANT PLANT GENE AND USE THEREOF

(71) Applicants: Zuhua He, Shanghai (CN); Hui Shen, Shanghai (CN); Qun Li, Shanghai (CN)

(72) Inventors: Zuhua He, Shanghai (CN); Hui Shen, Shanghai (CN); Qun Li, Shanghai (CN)

(73) Assignee: SHANGHAI INSITITUTE FOR BIOLOGICAL SCIENCES, CHINSE ACADEMY OF SCIENCES, Shanghai (CN)

(21) Appl. No.: 14/236,710

(22) PCT Filed: Aug. 6, 2012
(86) PCT No.: PCT/CN2012/079710

§ 371 Date: Jun. 9, 2014

(30) Foreign Application Priority Data
Aug. 5, 2011 (CN) 201110224902.X

(51) Int. Cl. C12N 15/82; G01N 33/53; C12Q 1/68; A01N 37/46

(52) U.S. Cl. CPC C12N 15/8261; A01N 37/46; C12N 15/8273; G01N 33/53; C12Q 1/6895
USPC 800/290; 504/335; 800/298; 436/501; 435/6.12

(57) ABSTRACT

Provided are a high temperature resistant plant gene and use thereof. The high temperature resistant gene can not only be used to modify she high temperature resistant property of a plant, but also has the functions of promoting plant growth, improving plant yield and increasing plant biomass and the like. The gene can also be utilized in the field of plant breeding to cultivate fine seed strain.

### TECHNICAL FIELD

The present invention relates to the fields of biotechnology and botany, more particularly, to a high temperature resistant plant gene and use thereof.

### BACKGROUND ART

Frequent high temperature stress brought by global climate changes is one of the major abiotic stresses, which seriously affects the growth and development as well as crop yield of plants, is a pressing issue facing sustainable agriculture.

Currently, the optimum temperature for the growth of many plants is 15-28° C. Too cold or too hot weather tends to affect plant growth and development substantially, especially to those with less tolerance to high temperature. For some plants suitable for human eating or viewing, however, it is necessary to ensure balanced growth or supply all the year round to meet the needs of daily life. Thus, screening excellent heat-resistant and stress-resistant plant variety by breeding or other biological means is particularly important.

Given that traditional breeding methods have low success rate and are time-consuming, it's also important to develop some new breeding techniques. Now, better technology in the present field is to modify or screen plant by analyzing and identifying stree-resistant related plant gene. Although some stree-resistant (e.g. heat-resistant) related genes have already been identified so far, we still need a lot of effort to further develop and find new heat-resistant gene, in order to provide more and better ways to improve plant varieties.

### CONTENTS OF INVENTION

The purpose of the invention is to provide a plant heat-resistant gene and use thereof.

In the first aspect, the present invention provides the use of ERECTA (for short, ER) protein or polynucleotide encoding thereof in improving heat resistance (heat-resistant, high temperature resistant or high temperature tolerance) ability of plants; promoting plant development; increasing plant yield; increasing plant biomass; reducing plant stomatal density; or improving plant water use efficiency (instantaneous).

In another preferred embodiment, said promoting plant development; increasing plant yield; increasing plant biomass include:

promoting plant leaves (including: cotyledon and leaf) to enlarge (including increasing the length and/or width of blade);

increasing plant petiole to become long;

promoting plant cells (including: epidermal cells and mesophyll cells) to become larger;

promoting plant bolting (including: increasing the number and/or branching number of the side of the moss and/or main moss);

increasing the number of plant inflorescence.

In another preferred embodiment, said heat resistance means the tolerable temperature of plant is greater than (including equal to) 28° C.; more particularly, the tolerable temperature is greater than 30° C.; more particularly, the tolerable temperature is greater than 35° C.; more particularly, the tolerable temperature is greater than the 40° C.

In another preferred embodiment, said ERECTA protein or the polynucleotide encoding thereof is used for the preparation of a plant with improved heat resistance (heat-resistant, high temperature resistant, high temperature tolerance) ability, rapid development, increased yield, increased biomass or low stomatal density.

In another preferred embodiment, said plant is selected from the group consisting of (but not limited to): Cruciferae, Gramineae or Solanaceae.

In another preferred embodiment, said plant is selected from the group consisting of (but not limited to): Arabidopsis thaliana, oilseed rape, Chinese cabbage, Little cabbage, beet of Cruciferae; rice, wheat, barley, maize, rye, sorghum, soybean of Gramineae; tomato (tomato), pepper, potato, tobacco, wolfberry, belladonna of Solanaceae.

In another preferred embodiment, said ERECTA protein is derived from Cruciferae plants (such as Arabidopsis thaliana, oilseed rape), Solanaceae plants (such as tomato), Gramineae plant (such as rice, corn, wheat, barley).

In another preferred embodiment, said ERECTA protein is derived from Arabidopsis thaliana.

In another preferred embodiment, the ERECTA protein is:

(a) a protein with amino acid sequence as set forth in SEQ ID NO: 3; or

(b) a protein derived from (a) by substitution, deletion or addition of one or more (e.g. 1-20; preferably 1-10; more preferably 1-5) residues in the amino acid sequence of SEQ ID NO: 3 and having the ability to improve plant heat resistance; or

(c) a polypeptide, having more than 70% (preferably more than 80%; more preferably greater than 90%; more preferably greater than 95%; more preferably greater than 99%) identity to the amino acid sequence defined in (a) and having the ability to improve plant heat resistance; or

(d) a protein fragment of SEQ ID NO: 3 and having the function of (a) protein (preferably having greater than 70%; more preferably greater than 75%; more preferably greater than 80%; more preferably greater than 85%; more preferably greater than 90%; more preferably greater than 95%; more preferably greater than 98% or 99% sequence identity with SEQ ID NO: 3).

In another preferred embodiment, the polynucleotide encoding ERECTA protein is:

(i) a polynucleotide having a sequence as set forth in SEQ ID NO: 1;

(ii) a polynucleotide, the nucleotide sequence of it can hybridize with polynucleotide sequence defined in (i) under stringent conditions and encoding a protein having the function to improve plant heat resistance;

(iii) a polynucleotide, the nucleotide sequence of it has more than 70% (preferably more than 80%; more preferably greater than 90%; more preferably greater than 95%; more preferably greater than 99%) identity with nucleotide sequence defined in (i) and encoding a protein having the function to improve plant heat resistance;

(iv) a polynucleotide, having sequence complementary to the sequence as set forth in SEQ ID NO: 1.

In another preferred embodiment, the polynucleotide encoding ERECTA protein is:

(i′) a polynucleotide having a sequence as set forth in SEQ ID NO: 2;

(ii′) a polynucleotide, the nucleotide sequence of it can hybridize with polynucleotide sequence defined in (i′) under stringent conditions and encoding a protein having the function to improve plant heat resistance;

(iii′) a polynucleotide, the nucleotide sequence of it has more than 70% identity with nucleotide sequence defined in (i′) and encoding a protein having the function to improve plant heat resistance; or

(iv′) a polynucleotide, having sequence complementary to the sequence as set forth in SEQ ID NO: 2.

In another aspect, the present invention provides a method for improving heat-resistant ability of plants, promoting plant development, improving plant yield, increasing plant biomass, reducing stomatal density or improving (instantaneous) water use efficiency of plants, said method comprises: improving the expression or activity of ERECTA protein in plants.

In another preferred embodiment, said method comprises: transferring the polynucleotide encoding ERECTA protein to the plant.

In another preferred embodiment, said method comprises the steps of:

(i) providing an agrobacterium strain containing an expression vector containing a polynucleotide encoding ERECTA protein;

(ii) contacting a plant cell, tissue or organ with the agrobacterium strain of step (i), so that said polynucleotide encoding ERECTA protein is transferred to the plant.

In another preferred embodiment, the method further comprises:

(iii) selecting the plant cell, tissue or organ transferred with the polynucleotide encoding ERECTA protein; and

(iv) regenerating the plant cell, tissue or organ of step (iii) and selecting the transgenic plants.

In another aspect, the present invention provides a plant with heat-resistant ability, rapid development, increased yield, increased biomass, low stomatal density or high (instantaneous) water use efficiency, which is a transgenic plant prepared by the foregoing method.

In another aspect, the present invention provides use of ERECTA protein or the polynucleotide encoding thereof for serving as a molecular marker to identify heat-resistant ability, development conditions, production, biomass, stomatal density or (instantaneous) water use efficiency of plants.

In another aspect, the present invention provides a method for identifying heat-resistant ability, development conditions, production, biomass, stomatal density or (instantaneous) water use efficiency of plants, the method comprises: detecting ERECTA protein expression in plant to be tested; if the expression of the polypeptide in plant to be tested is higher than (preferably statistically higher than e.g., over 20%; more preferably higher than over 50%; more preferably higher than over 80%) normal value (average) of ERECTA protein expression in the plant, said plant is the plant having heat-resistant ability, good development, high-yield, high biomass or low stomatal density; if the expression of the polypeptide in plant to be tested is less than (preferably statistically less than e.g., over 20%; more preferably less than over 50%; more preferably less than over 80%) normal value (average) of ERECTA protein expression in the plant, said plant is the plant not having heat-resistant ability and having low-yield, low biomass or high stomatal density.

The other aspects of the present invention will be apparent to the skilled person based on the contents disclosed herein.

### BRIEF DESCRIPTION OF THE DRAWINGS

FIG. 1. Overexpression of ERECTA promoted the development of Arabidopsis plants in rosette stage.

(A) The expression of ERECTA gene in wild type (Col-0) and the two overexpressing transgenic lines L2-3 and L7-1 determined by realtime PCR. Biologically repeated three times and the results were shown as mean±SD.

(B) Phenotype of seedlings growing in ½ MS medium for 10 days. Bar=1 mm.

(C) Phenotype of rosette stage seedlings after soil culture for 20 days. The left side of the row is overall observation, the right side of the row is the seedling rosette leaves (not including cotyledons) cutting in the petiole base in accordance with the order of growth successively, arranged from left to right. Bar=1 cm.

FIG. 2. Overexpression of ERECTA affected leaf development by promoting cell elongation.

(A) As shown in figure, morphological phenotype of ninth rosette leaf of seedlings after soil culture for 20 days. Bar=5 mm.

(B) Schematic diagram of semi-thin section of the blade cross-section.

(C)-(D), Statistical measurement value of length of the blade (C), width of the blade (D). The results were shown as mean±SD, ** means P<0.01, n=20.

FIG. 3. Overexpression of ERECTA promoted the development of side moss of plant.

(A) Morphology photos of seedlings of Overexpression line after soil culture for 30 days. Bar=2 cm.

(B) Measurement statistics of average inflorescence number in individual plant. The results were shown as mean±SD, ** means P<0.01, n=20; The unit of the ordinate value is “number of inflorescence”.

FIG. 4. Overexpression of ERECTA led to reduced stomatal density.

(A) Scanning electron micrographs of abaxial stomata distribution of Col-0, er-105, 35S::ERECTA L2-3, 35S::ERECTA L7-1 mature rosette leaves.

(B)-(C). Statistical measurement value of stomatal density (B), stomatal coefficient (C). The results were shown as mean±SD, n=25. In B, the unit of the ordinate value is number/mm²; In C, the ordinate value is a ratio, calculated by: the number of stomata within a blade region/(the number of stomata+the number of epidermal cells). It is a measurement indicator of stomatal development of leaf.

FIG. 5. The ERECTA-overexpressing plants had high temperature stress resistance at 40° C.

(A) Phenotype of Col-0, er-105, ERECTA-overexpressing lines L2-3 and L7-1 treated at 40° C. for 48 h after soil culture for 2 weeks. From left to right were, in order, wild-type Col-0, er-105, 35S::ERECTA L2-3, L7-1. Bar=1 cm.

(B) Statistics of survival rate of high temperature stress. Rehydrated for 2-3 days after high temperature treatment, wilting to yellow was considered as dead plant, plant retaining verdure was considered as survivable plant. Results were repeated for three times. The results were shown as mean±SD, * means P<0.05, ** means P <0.01, n=20.

FIG. 6. Conductivity measurement of wild-type, er mutant and overexpressing plants under high temperature stress.

Electrical conductivity measurement of Col-0, er-105, 35S::ERECTA L2-3, L7-1 at 0 h, 12 h, 24 h, 36 h and 48 h of high-temperature stress. The results showed the ratio of determined conductivity and total conductivity (ion leakage). n=10, biologically repeated three times.

FIG. 7. ERECTA-overexpressing lines had high temperature stress resistance at 30° C.

(A) The soil culture seedlings grew at 21° C. for 3 days, moved to 30° C. for heat treatment, observed phenotype after 30 days.

(B) The heat treated plants were rehydrated for 2-3 days, statistically analyzed survival rate. n=20, the treatment was repeated three times. The results were shown as mean±SD, * means P<0.05.

FIG. 8. The map of plasmid 35S-C1301.

FIG. 9. Phylogenetic chart of sequence homology based on the ERECTA (abbreviated as ER) and ERECTA-like (abbreviated as ERL) gene kinase domain.

Analyses utilizing neighbor-joining method and maximum parsimony of the PAUP software were used to detect homology and evolutional correlation (black value (located above the horizontal line) representing homology, red value (located below the horizontal line) representing evolutional correlation), respectively. Homologous genes of ERECTA were based on ERECTA (At2g26330, group B) and ERECTA-Like (At5g62230 and At5g07180, group A). Among which, At (Arabidopsis thaliana), Bo (Brassica oleracea L.), Eg (Palm), GM (soybean), HV (barley), Le (tomato), Os (rice), Sb (sorghum), SO (sugar cane), Ta (wheat), Zm (corn).

FIG. 10. Overexpression of ERECTA improved transpiration efficiency Arabidopsis.

Determination of transpiration efficiency of Col-0, 35S::ERECTA L7-1 under short-day growth conditions (8 hours in light). Results shown were the ratio of the maximum photosynthetic rate and transpiration rate.

Electrical conductivity measurement of Col-0, er-105, 35S::ERECTA L2-3, L7-1 at 0 h, 12 h, 24 h, 36 h and 48 h of high-temperature stress. The results shown were the ratio of determined conductivity and total conductivity (ion leakage). n=10, biologically repeated three times.

FIG. 11. Tomato ERECTA-overexpressing line had larger blade and improved heat resistance compared with wild type.

(A) Leaf morphological phenotype of T0 generation of ERECTA-overexpressing transgenic tomato (35S::ERECTA) after 4-week soil culture at 25° C. and no-load control.

(B) The top cutting seedlings of transgenic tomato plants (including ERECTA-overexpressing (35S::ERECTA) and no-load control) grew at 25° C. for 2 weeks, 45° C. treated for 72 hours, and observed the phenotype.

(C) The top cutting seedlings of tomato transgenic plants (including the ERECTA-overexpressing (35S::ERECTA) and no-load control) grew at 25° C. for 2 weeks 45° C. treated for 3 days, rehydrated for 2 days and observed the phenotype.

FIG. 12. Oilseed rape ERECTA-overexpressing line improved heat resistance compared with wild type.

T0 generation of transgenic oilseed rape (35S::ERECTA and no-load) was soil culture at 25° C. for 4 weeks, then perform high temperature treatment (left) and observed the phenotype. Rehydrated for 2 days at room temperature (25° C.) after the high temperature treatment, and observed the phenotype (right).

### DESCRIPTION OF THE PREFERRED EMBODIMENTS

After in-depth studies, the present inventor has discovered a new plant heat-resistant gene-ERECTA, and its protein. The present invention also discloses the use of this heat resistance gene, in particular for the improvement of plant traits of extreme high temperature resistance, while improving plant growth. ERECTA gene can be used in plant cultivation, breeding varieties with specific quality traits.

There is no specific limitation on the plants that can be used in the present invention, as long as they are suitable for gene transformation operations. The plants include various crops, flower plants or plants of forestry, etc. Specifically, the plants include, but are not limited to, dicotyledon, monocotyledon or gymnosperm. More specifically, the plants include, but is not limited to, wheat, barley, rye, rice, corn, sorghum, beet, apple, pear, plum, peach, apricot, cherry, strawberry, Rubus swinhoei Hance, blackberry, bean, lentil, pea, soy, rape, mustard, opium poppy, olea europea, helianthus, coconut, plant producing castor oil, cacao, peanut, calabash, cucumber, watermelon, cotton, flax, cannabis, jute, citrus, lemon, grapefruit, spinach, lettuce, asparagus, cabbage, Chinese cabbage, Little cabbage, carrot, onion, murphy, tomato, green pepper, avocado, cassia, camphor, tobacco, nut, coffee, aubergine, sugar cane, tea, pepper, grapevine, nettle grass, banana, natural rubber tree and ornamental plant, etc.

As a preferred embodiment, said “plant(s)” include, but are not limited to: Cruciferae, Gramineae, Rosaceae. For example, said “plant(s)” including, but not limited to: Cruciferae including Arabidopsis thaliana, oilseed rape, Chinese cabbage, Little cabbage, oilseed rape, sugar beet etc.; Gramineae including rice, wheat, barley, corn, rye, sorghum, soybean etc.; Solanaceae including tomato (tomato), pepper, potato, tomato, tobacco, wolfberry, belladonna.

As used herein, the “normal value (mean value) of ERECTA protein expression in such plants” is the “threshold” for determining ERECTA protein expression, those skilled in the art can easily obtain the normal value as ERECTA protein is a known protein. Method for comparing protein expression difference is also known, for example, by simple western blotting test.

As used herein, the term “heat resistance”, “resistance to heat”, “resistance to high temperature” or “high temperature tolerance” can be used interchangeably.

In the present invention, selecting suitable “control plant” is a routine part of the experimental design, and may include corresponding wild type plant or corresponding plant without target gene. Control plant is generally the same plant species or even the same variety as the plant to be assessed. The control plant may also be the individual losing transgenic plant due to separation. Control plant as used herein refers not only to whole plant, but also refers to the parts of the plant, including seeds and seed parts.

As used herein, the term “enhance”, “improve” or “increase” can be exchanged with each other and in the application, it shall mean compared with control plants as defined herein, at least 2%, 3%, 4%, 5%, 6%, 7%, 8%, 9% or 10%, preferably at least 15% or 20%, more preferably 25%, 30%, 35% or 40% or more improving in yield and/or growth and other useful agronomic traits.

The ERECTA protein of the present invention, a known protein in the art, is highly conserved in some plants. Its conservation in plant is shown in FIG. 9.

The ERECTA protein (polypeptide) of the present invention may be a recombinant polypeptide, a natural polypeptide, a synthetic polypeptide. The polypeptide of the present invention may be a naturally purified product, or a product of chemical synthesis, or produced from a prokaryotic or eukaryotic host (e.g., bacteria, yeast, higher plant, insect and mammalian cells) using recombinant techniques. According to the host used in the recombinant production protocol, the polypeptide of the present invention may be glycosylated or may be unglycosylated. The polypeptide of the present invention may also include or not include the starting methionine residue.

The present invention also includes ERECTA protein fragments, derivatives and analogs. As used herein, the term “fragment”, “derivative” and “analog” refers to the polypeptide substantially maintaining the same biological function or activity as ERECTA protein of the present invention. The fragments, derivatives or analogs of the polypeptide of the present invention, may be (i) a polypeptide in which one or more conservative or non-conservative amino acid residues (preferably a conserved amino acid residue) are substituted, and such substituted amino acid residues may or may not be encoded by the genetic code, or (ii) a polypeptide in which one or more amino acid residues have a substituent group, or (iii) a polypeptide formed by fusing mature polypeptide with another compound (for example, a compound prolonging the half-life of the polypeptide, e.g. polyethylene glycol), or (iv) a polypeptide formed by fusing additional amino acid sequence to the sequence of this polypeptide (such as leader sequence or secretory sequence or sequence used to purify the polypeptide or fibrinogen sequence, or fusion protein). According to the definitions herein, these fragments, derivatives and analogs are known to a person skilled in the art.

Any biologically active fragment of ERECTA protein can be applied to the present invention. Here, biologically active fragment of ERECTA protein means as a polypeptide, it is still able to maintain the whole or partial function of full-length ERECTA protein. Normally, said biologically active fragment is to maintain at least 50% activity of full-length ERECTA protein. Under the preferred conditions, said active fragment is able to maintain 60%, 70%, 80%, 90%, 95%, 99%, or 100% activity of full-length ERECTA protein.

In the present invention, the term “ERECTA protein” refers to the polypeptide of SEQ ID NO: 3 having the activity of ERECTA protein. The term also includes variant forms of SEQ ID NO: 3 having the same function as ERECTA protein. These variant forms include (but are not limited to): deletion, insertion and/or substitution of a plurality of (generally 1-50, preferably 1-30, more preferably 1-20, most preferably 1-10, further more preferably 1-8 or 1-5) amino acids, and addition or deletion of one or more (generally 1-50, preferably 1-30, more preferably 1-20, most preferably 1-10, further more preferably 1-8 or 1-5) amino acids at the C-terminus and/or N-terminus (particularly, N terminus). For example, in the art, when the substitution is carried out by amino acids with similar properties, or similar amino acids, the function of the protein is usually not changed. As another example, adding one or more amino acids at the C-terminus and/or N-terminus (particularly, N terminus) usually does not change the function of the protein. The term also includes active fragment and active derivative of ERECTA protein.

Variant forms of the polypeptide include: homologous sequences, conservative variants, allelic variants, natural mutants, induced mutants, the protein encoded by DNA which can hybridize with DNA of ERECTA protein under high or low stringency conditions, as well as the polypeptide or protein obtained by utilizing anti-serum of ERECTA protein. The present invention also provides other polypeptides, such as the fusion protein containing ERECTA protein or fragment thereof.

Any protein having high protein homology with said ERECTA protein (for example, having 50% or higher, preferably 60% or higher, preferably 70% or higher; preferably 80% or higher; more preferably 90% or higher, such as a homology of 95%, 98% or 99% homology with sequence as set forth in SEQ ID NO: 3) and having the same function as ERECTA protein is also included in the present invention. These proteins include, but are not limited to: ZmERECTA A. ZmERECTA B derived from corn Z; OsERECTA A, OsERECTA B derived from rice; SbERECTA A, SbERECTA B, SbERECTA C derived from two-color sorghum; GmERECTA A, GmERECTA B, GmERECTA C, GmERECTA D derived from soybean (see patent CN 101 589 147 or WO 2008039709 A2 or U.S. 2011/0,035,844 A1 for sequence).

The present invention also provides ERECTA protein or polypeptide analogs. The difference between these analogs and natural ERECTA protein can be a difference in amino acid sequence, also can be a difference not affecting modified forms of the sequence, or both. These polypeptides include natural or induced genetic variants. The induced variants can be obtained by a variety of techniques, such as generating random mutagenesis by irradiation or exposure to a mutagenic agent, but also by directed mutagenesis or other known molecular biology techniques. Analogs also include analogs having residues different from natural L-amino acid (e.g., D-amino acid), as well as analogs having non-naturally occurring or synthetic amino acids (such as β, γ-amino acids). It should be understood that the polypeptide of the present invention is not limited to the above-exemplified representative polypeptide.

Modification (normally not change the primary structure) forms comprise: a form of in vivo or in vitro chemical derivatization of polypeptides, such as acetylated or carboxylated. The modifications also include glycosylation. The modified forms also include a sequence having phosphorylated amino acid residues (e.g. phosphotyrosine, phosphoserine, phosphorylated threonine). Also included are polypeptides which are modified to have an improved anti-proteolysis property or optimize the solubility property.

In the present invention, “Conservative variant polypeptide of ERECTA protein” refers to a polypeptide having up to 20, preferably up to 10, more preferably up to 5, most preferably up to 3 amino acids in the amino acid sequence of SEQ ID NO: 3 being replaced by the amino acids with similar or close property. These conservative variant polypeptides are preferably produced by amino acid substitutions in accordance with Table 1.

TABLE 1

| Amino acid residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Vat; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also relates to a polynucleotide sequence encoding ERECTA protein of the present invention or its conservative variant polypeptide. Said polynucleotide may be in the form of DNA or RNA. The DNA includes cDNA, genomic DNA or artificially synthesized DNA. DNA may be single-stranded or double-stranded. DNA may be the coding strand or non-coding strand. The coding region sequence encoding mature polypeptide can be the identical or degeneration variant of coding region sequence as set forth in SEQ ID NO: 1 or SEQ ID NO: 2. As used therein, “a degeneration variant” refers to a nucleic acid molecule that encodes a protein having the sequence of SEQ ID NO: 3 with a nucleotide sequence different from the coding sequence as set forth in SEQ ID NO: 1 or SEQ ID NO: 2. Preferable, ERECTA genomic sequence (SEQ ID NO: 1 contains both exons and introns) or a variant of this sequence (including the degeneration variant thereof) is used to improve heat-resistant (resistant to heat, resistant to high temperature or high temperature-resistant) ability of plant; promote plant development; improve plant production; increase plant biomass; reduce plant stomatal density; improve (instantaneous) water use efficiency of plant.

Polynucleotides encoding mature polypeptide of SEQ ID NO: 3 comprise: coding sequence only encoding mature polypeptide; coding sequence of mature polypeptide, and various additional coding sequences; coding sequence of mature polypeptide (and optionally additional coding sequence) and non-coding sequence.

The term “polynucleotide encoding a polypeptide” may be a polynucleotide comprising sequence encoding said polypeptide, and also can be a polynucleotide including additional coding and/or non-coding sequence.

The present invention also relates to variants of above-mentioned polynucleotides which encode polypeptides or polypeptide fragments, analogs and derivatives having the same amino acid sequences as the present invention. These polynucleotide variants can be naturally occurring allelic variants or non-naturally occurring variants. These nucleotide variants include substitution variants, deletion variants, and insertion variants. As known in the art, allelic variant is an alternate form of polynucleotide, it may be one or more nucleotide substitutions, deletions or insertions, but will not substantially alter its polypeptide-encoding function.

The present invention also relates to a polynucleotide hybridizing to any of the above sequences and having at least 50%, preferably at least 70%, more preferably at least 80% sequence identity between the two sequences. The present invention specifically relates to a polynucleotide hybridizing to the polynucleotides of the present invention under stringent conditions. In the present invention, the “stringent condition” refers to: (1) hybridization and elution at a relatively lower ionic strength and relatively higher temperature, such as 0.2×SSC, 0.1% SDS, 60° C.; or (2) presence of denaturation agent during hybridization, such s 50% (v/v) formamide, 0.1% calf serum/0.1% Ficoll, 42° C., and the like; or (3) conditions only allowing hybridization between two sequences that have at least 90%, preferably at least 95% identity. Moreover, the polypeptide encoded by the hybridizing polynucleotide exhibits the same biological function and activity as those of the mature polypeptide as set forth in SEQ ID NO: 3.

The present invention also relates to a nucleic acid fragment hybridizing with the above-described sequence. As used herein, “nucleic acid fragment” contains at least 15 nucleotides, preferably at least 30 nucleotides, more preferably at least 50 nucleotides, and most preferably at least 100 or more nucleotides in length. The nucleic acid fragment can be used for nucleic acid amplification techniques (e.g. PCR) to determine and/or separate the polynucleotide encoding ERECTA protein.

It should be understood that although ERECTA gene of the present invention is preferably from Gramineae, other genes obtained from other plants and highly homologous (e.g. having more than 80%, eg 85%, 90%, 95% or even 98% sequence identity) with ERECTA gene are also considered within the scope of the present invention. Methods and tools comparing sequence identity are well known in the art, for example BLAST.

The full-length nucleotide sequence of ERECTA protein of the present invention or fragments thereof can usually be obtained by PCR amplification method, recombinant method or artificial synthesis. As to PCR amplification method, the sequences of interests can be amplified by designing primers according to the related nucleotide sequence disclosed in the present invention, especially the open-reading frame, and using a commercially available cDNA library or a cDNA library prepared according to any of the conventional methods known in the art as a template. For an excessively long sequence, typically, two or more PCR amplifications are needed, and then, the fragments obtained in the amplifications are ligated together in a correct orientation.

The present invention also relates to a vector comprising said polynucleotide, as well as a host cell generated with said vector or ERECTA protein-coding sequence by genetic engineering.

In the present invention, ERECTA protein polynucleotide sequences can be inserted into a recombinant expression vector. The term “recombinant expression vector” refers to bacterial plasmid, bacteriophage, yeast plasmid, plant cell virus, mammalian cell virus, or other carriers well known in the art. In short, any plasmid and vector may be used as long as it can be replicated and stable in the host. An important feature of the expression vector is typically containing an origin of replication, promoter, marker gene and translation control elements.

Containing above-mentioned suitable DNA sequence and appropriate promoter or vector controlling sequence, can be used to transform an appropriate host cell, to allow for protein expression. The host cells can be prokaryotic cells, such as bacterial cells; or lower eukaryotic cells, such as yeast cells; or higher eukaryotic cells, such as plant cells. Representative examples include: E. coli, Streptomyces, Agrobacterium; fungal cells such as yeast; plant cells.

When said polynucleotide is expressed in higher eukaryotic cells, transcription will be improved if enhancer sequences are inserted in the vector. Enhancers are cis-acting factors of the DNA, usually about from 10 to 300 base pairs, and act on the promoter to enhance gene transcription.

The skilled in the art are aware of how to select an appropriate carrier, promoter, enhancer and host cell.

Transforming a host cell with recombinant DNA can be carried out by conventional techniques well known to those skilled in the art. Transformation of plant may also be achieved by using agrobacterium or gene gun transformation, and the like, such as spraying method, leaf disc method, rice immature embryo transformation etc.

The present invention provides uses of said ERECTA protein or encoding gene thereof, for improving high temperature-resistant ability of the plant; said ERECTA protein can also be used to: promote plant development; improve yield of plant; increase plant biomass; or reduce leaf stomatal density. Said promotion of plant development or increase in the yield of plant or biomass includes: promoting plant leaf (including: cotyledon and leaf) to enlarge (including increasing length and/or width of the blade); increasing petiole to become long; promoting plant cells (including: epidermal cells and mesophyll cells) to become larger; promoting plant bolting (including: increasing the number of side moss and/or main moss and/or the number of branches); raising the number of plant inflorescence. Or for screening substances useful for adjusting high temperature-resistant ability, development, yield or biomass, or stomatal density of the plant (i.e.: said substances adjust high temperature-resistant capability, development, yield, biomass, or stomatal density of the plant through regulating the expression of ERECTA protein).

The present invention also relates to ERECTA agonist or antagonist and its use. Since the agonist or antagonist of ERECTA can adjust ERECTA expression and/or adjust the activity of ERECTA, etc., therefore, the agonist or antagonist of said ERECTA may also regulate high temperature-resistant capability, development, yield, biomass, or stomatal density of plant by affecting ERECTA, so as to achieve the purpose of improving plant.

Any substance which can improve the activity of ERECTA protein, improve the stability of ERECTA protein, promote ERECTA protein expression, extend effective action time of ERECTA protein, or promote ERECTA transcription and translation may be used in the present invention, as a substance which can be used for improving high temperature-resistant ability of the plant, and promoting plant development, increasing plant yield or biomass or reducing stomatal density of the plant. Any substance which can reduce the activity of ERECTA protein, reduce the stability of ERECTA protein, inhibit ERECTA protein expression, decrease effective action time of ERECTA protein, or to reduce ERECTA transcription and translation may be used in the present invention, as the down-regulator, antagonist or inhibitor of ERECTA (i.e.: down-regulating substances expressed by ERECTA protein-coding gene), such as antibody of said ERECTA protein, interfering with interfering molecule expressed by said ERECTA protein-coding gene (e.g. interfering molecule which may form microRNA). Said down-regulator, antagonist or inhibitor can be used to reduce high temperature-resistant ability of the plant, inhibit plant development, reduce plant yield or biomass or increase stomatal density of the plant. After disclosure of the target sequence, method for preparing interfering molecule which interferes with the expression of specific genes is well known to those skilled in the art.

The present invention also relates to a method for improving plants, the method comprises adjusting ERECTA protein expression in said plant.

In one aspect, the present invention provides a method to improve high temperature-resistant ability of plant, promote plant development, increase plant yield or biomass, reduce stomatal density of plant or improve water use efficiency of plant, said method comprises: improving the expression or activity of ERECTA protein in the plant; or making said plant overexpress ERECTA protein.

In another aspect, the present invention also provides a method of reducing high temperature-resistant ability of plant, inhibiting plant development, reducing plant yield or biomass, or increasing leaf stomatal density of plant, said method comprises: reducing ERECTA protein expression in said plant, including no or low expression of ERECTA protein.

After knowing the uses of said ERECTA protein, a variety of methods well known to those skilled in the art can be used to adjust said ERECTA protein expression. For example, expression unit (for example, expression vector or virus, etc.) carrying ERECTA gene will be delivered to the target site by ways known to the skilled in the art, to make it express active ERECTA protein. In addition, it is also possible to employ a variety of methods well known to those skilled in the art to reduce the expression of ERECTA protein or make deletion expression, for example, expression unit (such as expression vector or virus, etc.) carrying antisense ERECTA gene will be delivered to the target site, making the cells or plant tissues not express or decrease express the ERECTA protein.

As one embodiment of the present invention, the gene encoding ERECTA protein is cloned into an appropriate vector by a conventional method, said recombinant vector with the exogenous gene is introduced into a plant cell which can express said ERECTA protein, so that the ERECTA protein is expressed. Plants overexpressing ERECTA protein can be obtained by regenerating said plant cell to plants.

Preferably, there is provided a process for preparing a transgenic plant, comprising:

(1) transferring a exogenous polynucleotide encoding ERECTA protein to a plant cell, tissue, organ or seed, obtaining the plant cell, tissue, organ or seed transferred with the polynucleotide encoding ERECTA protein; and

(2) regenerating the plant cell, tissue, organ or seed obtained in step (1) which was transferred with the exogenous polynucleotide encoding ERECTA protein into plants.

As a preferred example, said method comprises the steps of:

(s1) providing an agrobacterium strain carrying an expression vector, said expression vector contains a polynucleotide encoding ERECTA protein;

(s2) contacting the plant cell, tissue, organ with the agrobacterium strain in step (s1), thereby the polynucleotide encoding ERECTA protein is transferred into the plant cell and integrated into chromosome of the plant cell;

(s3) selecting the plant cell, tissue, organ or seed transferred with the polynucleotide encoding ERECTA protein; and

(s4) regenerating the plant cell, tissue, organ or seed in step (s3) into plants.

Other methods to increase the expression of ERECTA gene or its homologous gene are known in the art. For example, by use a strong promoter to drive, thereby enhance the expression of ERECTA gene or its homologous gene. Or enhance the ERECTA gene expression by enhancers (such as first intron of rice waxy gene, first intron of Actin gene, etc.). Strong promoters suitable for the method of the present invention include but are not limited to: the 35S promoter, Ubi promoter of rice, maize, etc.

As an optional embodiment, there is also provided a method for reducing ERECTA protein expression in plant, said method comprises:

(1) transferring an interfering molecule which interferes with ERECTA gene expression to a plant cell, tissue, organ or seed, obtaining the plant cell, tissue, organ or seed transferred with said interfering molecule; and

(2) regenerating the plant cell, tissue, organ or seed obtained in step (1) which was transferred with said interfering molecule into plants.

As a preferred example, said method comprises the steps of:

(i) providing an agrobacterium strain carrying a vector which can interfere with gene expression, said expression vector is selected from the group consisting of:
(a) a vector containing ERECTA protein-encoding gene or gene fragment starting in the opposite direction (antisense molecule);
(b) a vector containing interfering molecules which can form components specifically interfering with ERECTA protein-encoding gene expression (or transcription) in the plant;

(ii) contacting the plant cell, tissue or organ with the agrobacterium strain in step (i), thereby said vector is transferred to the plant cell, tissue or organ.

Preferably, the method further comprises:

(iii) selecting the plant cell, tissue or organ transferred with said vector; and

(iv) regenerating the plant cell, tissue or organ in step (iii) into plants.

Other methods for inhibiting the expression of ERECTA gene or its homologous gene are well known in the art.

The present invention also includes the use of plants obtained by any above-mentioned method, said plant comprises: transgenic plants transferred with ERECTA gene or its homologous gene; plants with reduced ERECTA protein expression (including low or no expression) and so on.

Any suitable conventional means, including reagent, temperature and pressure conditions, can be used to implement said method.

In addition, the present invention also relates to ERECTA protein or its encoding gene as a tracing marker for progeny of transformed plants. The present invention also relates to ERECTA protein or its encoding gene as a molecular marker. The high temperature-resistant performance, production level, stomatal density of the plants can be identified by the determination of ERECTA protein expression in the plants. Improved varieties of plant can be screened by using ERECTA protein or its encoding gene.

In a specific embodiment of the present invention, the present inventor used Arabidopsis thaliana ecotype Col-0 and Ler for QTL analysis, identified a gene involved in the high temperature stress, ERECTA (At2g26330). To explore the application prospects of ERECTA in plant resistance to high temperature stress, the present inventors constructed an ERECTA gene-overexpressing Arabidopsis lines driven by 35S promoter. The data show that the ERECTA gene overexpression not only gives the plant resistance to extreme heat stress (40° C.), but also enhances plant resistance to moderate heat stress (30° C.). Meanwhile, the cells of these transgenic plants are enlarged, resulting in the increase of various organs, increase of the biomass; while the number of stomata significantly reduced.

The present invention for the first time identifies high temperature-resistant QTL gene ERECTA, and performs analysis and evaluation of its application potential in high temperature-resistant molecular breeding. The datas of the present inventor show that ERECTA overexpressing lines aren't hindered in growth and development at the same time when acquiring stress resistance; on the contrary, overexpressing lines show increase in blade, increase in inflorescence, and a significant increase in biological yield. This shows the increase in ERECTA expression, to some extent, promotes plant development. Therefore, ERECTA gene can be used as a target gene to modify crops, expected to balance the relationship between crop yield and improving resistance.

The present invention will be further illustrated in combination with the following examples. It should be understood that these examples are for illustrating the present invention, but not for limiting the scope of the present invention. The experimental method in which the specific conditions are not specifically indicated in the following examples generally is performed according to the conventional conditions, such as those described in Sambrook et al., Molecular Cloning: A Laboratory Manual (New York: Cold Spring Harbor Laboratory Press, 2002), or according to the conditions recommended by the manufacturer. Unless otherwise specifically indicated, the percent and part are calculated based on weight.

Unless otherwise specifically indicated, all of the scientific terms used herein have the same meanings as those familiar to the skilled in the art. Further, any methods and materials equivalent to the disclosed contents can be used in the present invention. The preferred practicing method and material disclosed herein are just for illustrative purpose.

### I. MATERIALS AND METHODS

1. Materials

(1) Arabidopsis (Arabidopsis thaliand) ecotype: Columbia (Col-0).

(2) Strain: Agrobacterium (Agrobacterium tumefaciens), GV3101 (Invitrogen).

(3) Transgenic material: col-0 p35S::ERECTA.

2. Methods

2.1 Planting of Arabidopsis

For Arabidopsis sterile culture, the seeds were surface (70% ethanol, 30 seconds; washed 4 times in sterile water) and deep (7% sodium hypochlorite for 10 minutes; sterile water 3 times) disinfected, sown in ½ MS (½×Murashige and Skoog basal Medium, 0.8% agar powder, pH 5.8) solid medium, placed at 4° C. for 72 h, and then transferred to 22° C. culture. A week later, the seedlings were transplanted in artificial soil (vermiculite, black soil and perlite 3:1:0.5) soaked with nutrient solution (3 g/10 L Hua Wuque, Shanghai Yong Tong Chemical Co., Ltd.), and then turned to phytotron. Wherein the plants for genetic analysis were cultured in phytotron with a photoperiod of 14 hours light and 10 hours dark (14/10 (L/D)).

2.2 Stress Treatment

Observed and identified high temperature tolerance of transgenic plants and their control through extreme high temperature and moderate high temperature.

Normal growth temperature for Arabidopsis was 21-23° C. The temperature raised to 30° C. was medium high temperature stress (generally wild-type Col-0 can survive for about 30 days at 30° C.); temperature raised to 40° C. was extreme high temperature (generally wild-type Col-0 can survive for about 48 hours at 40° C.); The results would be more objective by treating with two degrees of high temperature stress.

2.2.1 Extreme High Temperature (40° C.)

Arabidopsis materials grew for 2-3 weeks in soil and transferred to the light incubator (MMM Climacell-111) for heat treatment. 30 individuals for each group, the same treatment were repeated three times. The treating conditions were 40° C., humidity 80%.

Treatment employed progressive processing method, that is, the temperature raised from 21° C.-30° C. for 12 hours—36° C. for 12 hours—40° C. The soil layer was more than or equal to 10 cm, to ensure adequate moisture during the treatment. After 24 hours of treatment (i.e.: observed after 40° C. treatment for 24 hours) or rehydration at room temperature for 2-3 days, observed the statistical survival rate of the Ler background materials. After 48 hours of treatment or rehydration at room temperature for 2-3 days, observed the statistical survival rate of col-0 background materials.

2.2.2 Moderate High Temperature (30° C.)

Arabidopsis seedlings grew for 3 days in soil and transferred to the 30° C. incubator for treatment, observed the phenotype after treatment for 30 days. After the treatment, rehydrated the plants for 2-3 days and calculated statistical survival rate. n=20, the treatment was repeated three times.

2.3 Arabidopsis Transformation

Spraying method was used for Arabidopsis transformation. 4-5 week-old plants growing well were used (cut the main moss a week before transformation, which will help the side moss to produce more bud, to improve transformation efficiency). Agrobacterium containing a gene transfer vector (p35S::ERECTA) was incubated at 28° C. to an OD₆₀₀ value between 1.2 to 1.4, centrifuged at 5,000 rpm for 10 min, the bacterial pellet was suspended in freshly prepared transfer solution (½ MS liquid medium containing 5% (w/v) sucrose, 0.03% (v/v) Silwet L-77 to a final concentration of OD₆₀₀≈0.6-0.8. Prior to transformation, removed pollinated flowers and seed pods and made the soil absorb enough water. When transformation, bacteria solution was evenly sprayed on Arabidopsis until the droplets were dropping from the leaves. The plants were covered with a black plastic bag to maintain humidity, in the dark overnight. After 24 hours, the plants were transferred to normal conditions. Until the seeds matured, the plants were mixed and harvested in paper bags, placed in a desiccator for 7 days and then subject to threshing. After disinfection, the seeds of T1-generation were sown in ½ MS medium containing 30 pg/ml Hygromycin B for screening. Seedlings showing no resistance to Hygromycin B can't grow normally and was very short. While positive seedlings carrying the gene transfer vector showed that hypocotyl and root can elong normally and cotyledons were large.

2.4 Vector Construction

p35S::ERECTA

Constructed a plant expression vector based on vector pCambia 1301 (www.cambia.org.au). First, NOS terminator sequence (see The Plant Journal (2001) 27 (2), 101-113) was introduced into EcoRI/PstI sites of pCambia 1301, to obtain C1301Nos3′. Then a 0.9 kb CaMV 35S promoter fragment (see The Plant Journal (2001) 27 (2), 101-113) was connected into C1301Nos 3′ using PstI and KpnI sites to obtain 35S-C1301, also introduced other multi-cloning sites, specific distribution of the sites was shown in FIG. 8.

ERECTA full-length genome sequence (including the 5′UTR and 3′UTR) was divided into two sections, anterior section (5′ end fragment) was obtained by PCR amplification, posterior section (3′ end fragment)) was obtained by enzyme digestion of BAC. Specific steps were as follows: BAC T1D16 was subject to double digestion with EcoRI and SnaBI, recycled about 7.8K fragment, i.e., posterior section of ERECTA full-length genome sequence (3′ end fragment); connected with pBluescript II SK (commercially available from Stratagene) digested by EcoRI and Sma1. Confirmed correct clone was connected to pCambia 1301 (www.cambia.org.au) after double digestion with KpnI and BamHI, to constitute pERECTA::ERECTA.

BAC T1D16 (available from Arabidopsis Biological Resource Center http://www.arabidopsis.org; Accession NO. 2585430) was used as a template, performed PCR amplification (primer 5′-tATCGATgtatatctaaaaacgcagtcg-3″ (SEQ ID NO: 4); 5′-aatatttgtcagttcttgagaag-3′ (SEQ ID NO: 5) to obtain the 412 bp ERECTA genomic DNA 5′ end fragment, and introduced ClaI restriction site to its 5′ end. The obtained sequence was confirmed by sequencing and connected to ClaI/SphI-digested pERECTA::ERECTA. The above-obtained recombinant plasmid was subject to ClaI/BamHI double digestion to obtain ERECTA full length genomic sequence (SEQ ID NO: 1), connected to the 35S-C1301, obtained p35S::ERECTA.

2.5 Determination of Conductivity

At each timepoint in the process, 5 plants with soil culture for about two weeks were selected for each genotype, pick 15 newly generated mature leaves from the said 5 plants, each leaf divided into two along the midrib, all samples were equally divided into 10 parts, placed in 3 ml of deionized water, respectively, shook in 28° C. shaker overnight. Electric conductivity meter (Mettler toledo, FE30) was used to determine conductivity ILᵢ; then the samples were treated with high temperature and pressure for 10 minutes, measured conductivity ILₜ until the temperature of the sample decreased to room temperature; relative conductivity was calculated in accordance with the following formula.

Ion leakage(Ion leakage)=ILᵢ/ILₜ×100% Formula 1

2.6 Observation and Statistics of Stomatal Density

Scanning electron microscopy was used to observe abaxial side of mature rosette leaves, five regions for each blade were observed, and at least five blades were observed. The number of pores per square millimeter area was calculated, as the measurement value of stomatal density.

2.7 Observation and Statistics of Stomatal Coefficient

Scanning electron microscopy was used to observe abaxial side of mature rosette leaves, five regions for each blade were observed, and at least five blades were observed. Stomatal coefficient (Stomata index, SI) was calculated using the following equation:

SI=number of stomata/(number of stomata+number of epidermal cells)×100% Formula 2

2.8 Loss-of-Function Mutant of ERECTA er-105

The mutants were obtained from Arabidopsis Biological Resource Center http://www.arabidopsis.org (cs89504).

Detailed construction method see reference The Plant Cell, Vol 8, 735-746, April, 1996.

2.9 Gene Transfer of Rape

1. Rape seed was soaked in 75% alcohol for 30 seconds, and then soaked with 0.1% mercuric chloride solution for 10 minutes, then rinsed with sterile water, sterilized seeds were plated on ½ MS medium and cultured at 25° C.1° C. for 4-7 days, light intensity was 80 μmol/m²·s.

2. Aseptic seedlings of rape after 4-7d culture were cut with a scalpe to get petiole, inoculated to ½ MS medium containing 1 mg/L 2, 4-D for 2 days.

3. After 2d culture, the petiole was co-cultured with Agrobacterium with OD₆₀₀=0.5 (containing foregoing constructed p35S::ERECTA) for 30-60 sec, sucked up bacteria solution on the cotyledon petiole, co-incubated for 1-2 days (MS+1 mg/L 2, 4-D).

4. Transferred to the selection medium MS+3 mg/L 6−BA+0.15 mg/L NAA+2.5 mg/L AgNO₃+Cef 250-500 mg/l (or Carb: 250-500 mg/l)+screening gene carried by the vector itself.

5. Until resistant bud grew to 1-2 cm, cut from the base of the bud and inserted into ½ MS rooting medium to induce rooting, adventitious roots of regeneration seedlings grew into seedlings after 2-3 weeks, opened the bottle and performed hardening-seedling for 2 d.

6. Washed away agar of the roots until root formation, transferred to a small bowl filled with potting soil, covered with plastic film for moisturizing 1-2 d and then removed the film. After two weeks of culture, the plants were moved into the field for normal cultivation and management.

2.10 Gene Transfer of Tomato

1. Test seeds were soaked in 70% alcohol for 1 minute, washed three times with sterile water. Soaked with 10% sodium hypochlorite solution for 5-10 minutes and rinsed 5-6 times in sterile water. The sterilized seeds were plated on ½ MS medium, and cultured at 26° C. for 7-9 days, light intensity was 80 μmol/m²·s, until the cotyledons grew. The cotyledons were cut and placed on ½ MS medium (containing glucose 30 g/L, NAA 1 mg/L, BAP 1 mg/L). The cotyledons were pre-incubated at 25° C. for 24 hours under low light conditions (10 μEm-2s-1).

2. Agrobacterium (containing foregoing constructed p35S::ERECTA) solution (containing 0.1 mM AS) with OD₆₀₀=0.5 was poured into a petri dish filled with the cotyledons, placed at room temperature for 30 minutes and then sucked up bacteria solution, co-incubated at 25° C. in the dark for 48 hours.

3. Co-cultured cotyledons were transferred to screening medium containing specific antibiotic (½ MS containing sucrose 30 g/L, Zeatin 1 mg/L, IAA 0.1 mg/L, Kan 0.1 g/L, Timentin 0.3 g/L) to culture.

4. After two weeks, callus with bud primordium was cut into small pieces, and transferred to subculture medium (½ MS containing sucrose 15 g/L, Kan 0.1 g/L, Timentin 0.3 g/L) to culture.

5. Until resistant bud grew to 2-4 cm, cut the bud from the bud base and inserted into rooting medium (½ MS containing IAA 5 mg/L, Kan 0.1 g/L, Timentin 0.3 g/L) to induce rooting, adventitious roots of regeneration seedlings grew into seedlings after 2 weeks, transplanted to soil.

2.11 Molecular Identification of Transgenic Positive Plants

Positive seedlings obtained from transgenic Arabidopsis, tomato, rape through resistance screening were further subject to molecular identification by PCR reaction. Collected new leaf tissues from the wild-type (Arabidopsis thaliana Col-0, tomato LA1589, rape Zheshuang 758) and the transgenic positive seedlings to perform routine DNA extraction. The extracted DNA was used as a template for the following procedures for PCR reaction: 94° C. 5 min; 94° C. 30 s—58° C. 30 seconds—72° C. 45 seconds, in total 30 cycles; Finally elongated at 72° C. for 10 minutes. The primers used 35S-F: 5′-GAACTCGCCGTAAAGACTG-3′ (SEQ ID NO: 6), ERECTA-663-R 5′-TGACTTCTTAATCTCCAGCAACG-3′ (SEQ ID NO: 7).

The electrophoresis results showed that the wild-type Arabidopsis, tomato, rape template DNA, as negative control of the PCR reaction, can't amplify bands; Arabidopsis, tomato and rape transgenic positive seedlings specifically amplified about 1.1 kb band; while non-positive seedlings can't amplify band as the wild-type.

2.12 Determination of Transpiration Efficiency

Measured the maximum photosynthetic rate (A) and transpiration rate (E) of the plant using photosynthesis analyzer (L1-6400) under the light intensity of 300 μmol m⁻²s⁻², CO₂ concentration of 400 mbar, leaf temperature of 22° C. The data was measured at 10: 30-11:30. Selected blades were newborn fully extended blades under short-day (8 hours of light), using 6-8 plants per line. Real-time leaf transpiration efficiency is calculated as A/E.

2.13 Determination of High-Temperature Resistance of Transgenic Tomato, Rape

For tomato T₀ generation transgenic plants (including no-load control and transgenic plants of ERECTA over-expressing vector (p35S::ERECTA)), the fourth leaf (including the stem of lower portion of the fourth leaf) from its tip was picked for cottage, the soil content of each cuttage plant was consistent. After two weeks of 25° C. culture, the materials were placed in the light incubator (MMM Climacell-111) to start high temperature treatment. Rape T₀ generation transgenic plants were directly subject to high-temperature treatment after growing for 30 days. The progressive processing method is processed: 30° C. 24 h→32° C. 24 h→36° C. 24 h→38° C. 24 h→43° C. 24 h, the final processing temperature was 45° C. During the processing, the watering was uniformed to maintain the consistency of the soil moisture content of each plant. After the end of the treatment, rehydrated at room temperature of 25° C. for 2-3 days, observed phenotype.

### II. EXAMPLE

### Example 1

### Gene Information

The present inventor used Arabidopsis ecotype Col-0 and Ler for QTL analysis, identified a gene involved in the high-temperature stress, ERECTA.

DNA sequence of ERECTA gene was as SEQ ID NO: 1; wherein the coding region sequence of ERECTA gene was as SEQ ID NO: 2.

The amino acid sequence of the protein encoded by the gene was as follows (SEQ ID NO: 3):

MALFRDIVLLGFLFCLSLVATVTSEEGATLLEIKKSFKDVNNVLYDWT
TSPSSDYCVWRGVSCENVTFNVVALNLSDLNLDGEISPAIGDLKSLLS
IDLRGNRLSGQIPDEIGDCSSLQNLDLSFNELSGDIPFSISKLKQLEQ
LILKNNQLIGPIPSTLSQIPNLKILDLAQNKLSGEIPRLIYWNEVLQY
LGLRGNNLVGNISPDLCQLTGLWYFDVRNNSLTGSIPETIGNCTAFQV
LDLSYNQLTGEIPFDIGFLQVATLSLQGNQLSGKIPSVIGLMQALAVL
DLSGNLLSGSIPPILGNLTFTEKLYLHSNKLTGSIPPELGNMSKLHYL
ELNDNHLTGHIPPELGKLTDLFDLNVANNDLEGPIPDHLSSCTNLNSL
NVHGNKFSGTIPRAFQKLESMTYLNLSSNNIKGPIPVELSRIGNLDTL
DLSNNKINGIIPSSLGDLEHLLKMNLSRNHITGVVPGDFGNLRSIMEI
DLSNNDISGPIPEELNQLQNIILLRLENNNLTGNVGSLANCLSLTVLN
VSHNNLVGDIPKNNNFSRFSPDSFIGNPGLCGSWLNSPCHDSRRTVRV
SISRAAILGIAIGGLVILLMVLIAACRPHNPPPFLDGSLDKPVTYSTP
KLVILHMNMALHVYEDIMRMTENLSEKYIIGHGASSTVYKCVLKNCKP
VAIKRLYSHNPQSMKQFETELEMLSSIKHRNLVSLQAYSLSHLGSLLF
YDYLENGSLWDLLHGPTKKKTLDWDTRLKIAYGAAQGLAYLHHDCSPR
IIHRDVKSSNILLDKDLEARLTDFGIAKSLCVSKSHTSTYVMGTIGYI
DPEYARTSRLTEKSDVYSYGIVLLELLTRRKAVDDESNLHHLIMSKTG
NNEVMEMADPDITSTCKDLGVVKKVFQLALLCTKRQPNDRPTMHQVTR
VLGSFMLSEQPPAATDTSATLAGSCYVDEYANLKTPHSVNCSSMSASD
AQLFLRFGQVISQNSE

### Example 2

### ERECTA Overexpression Promoted the Development of Rosette Stage Arabidopsis Plant

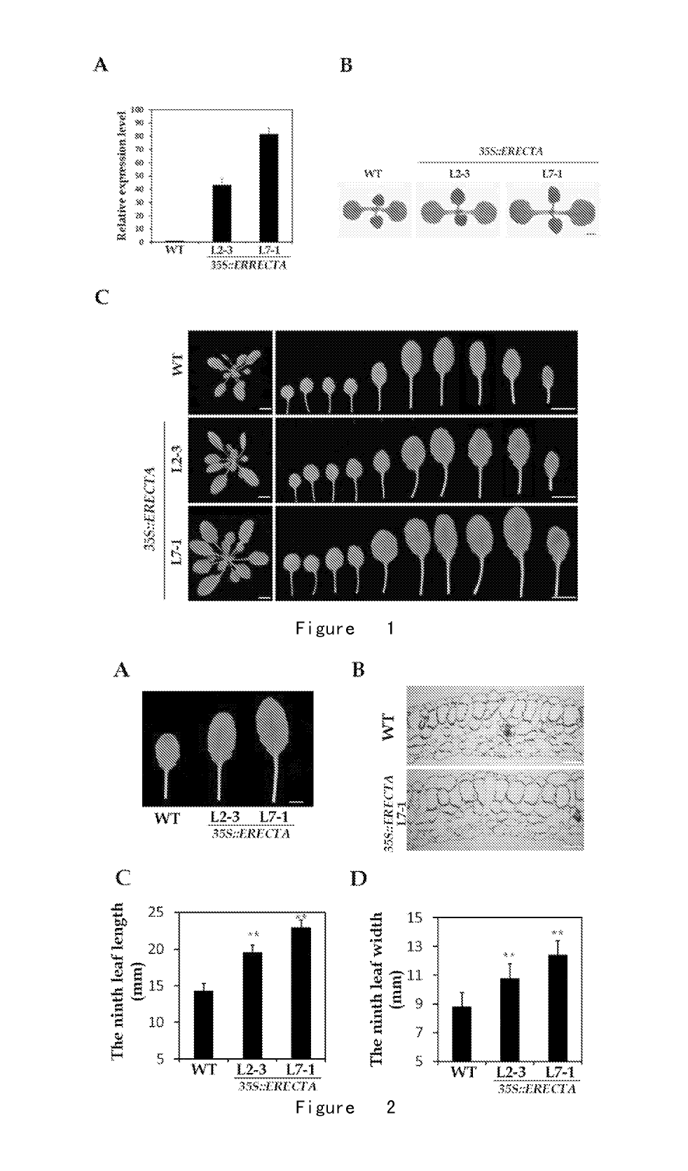

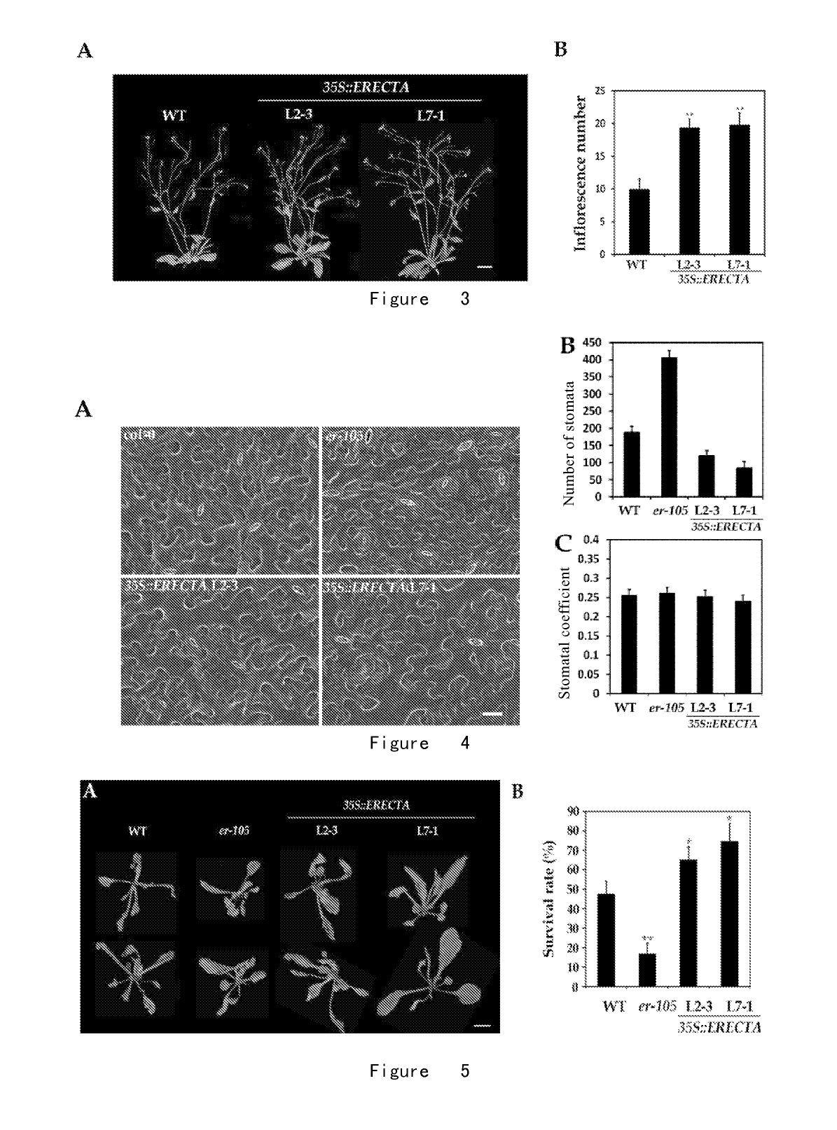

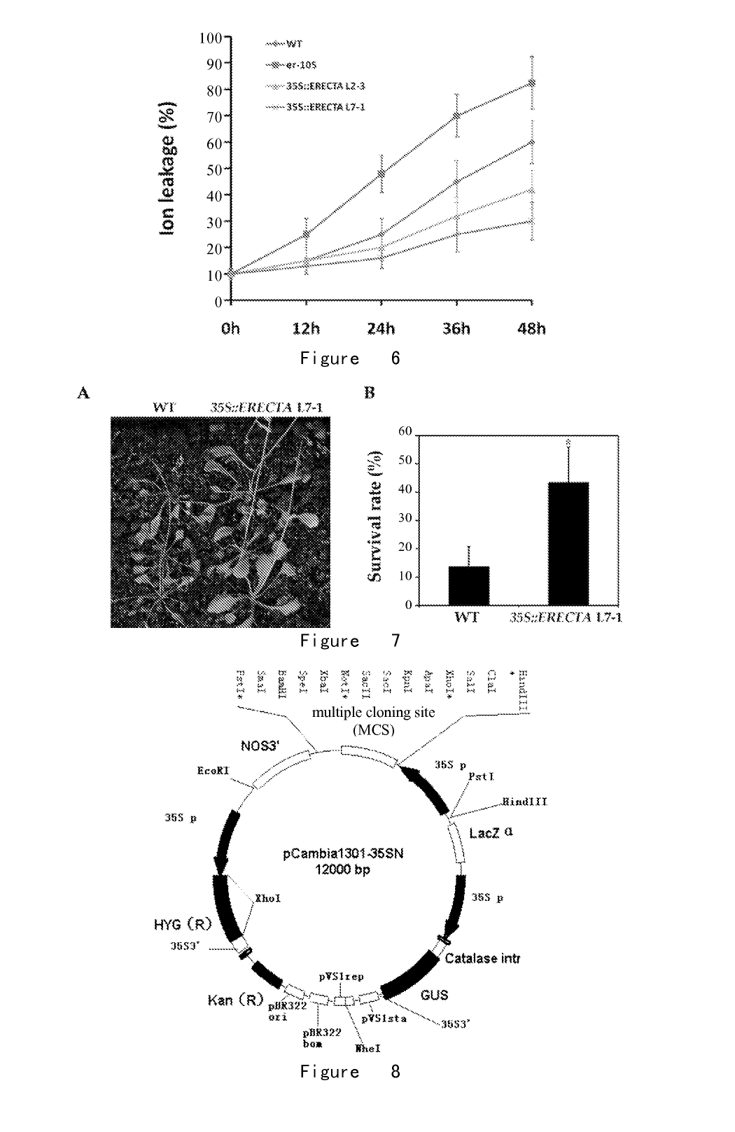

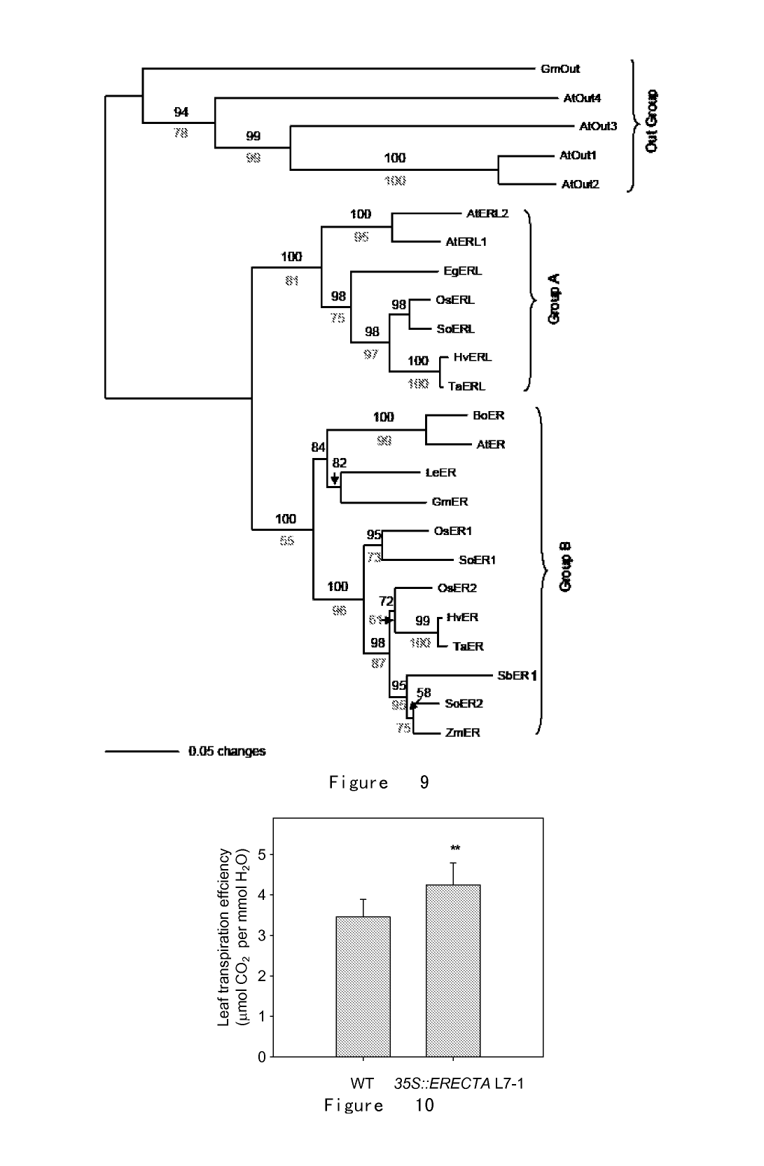

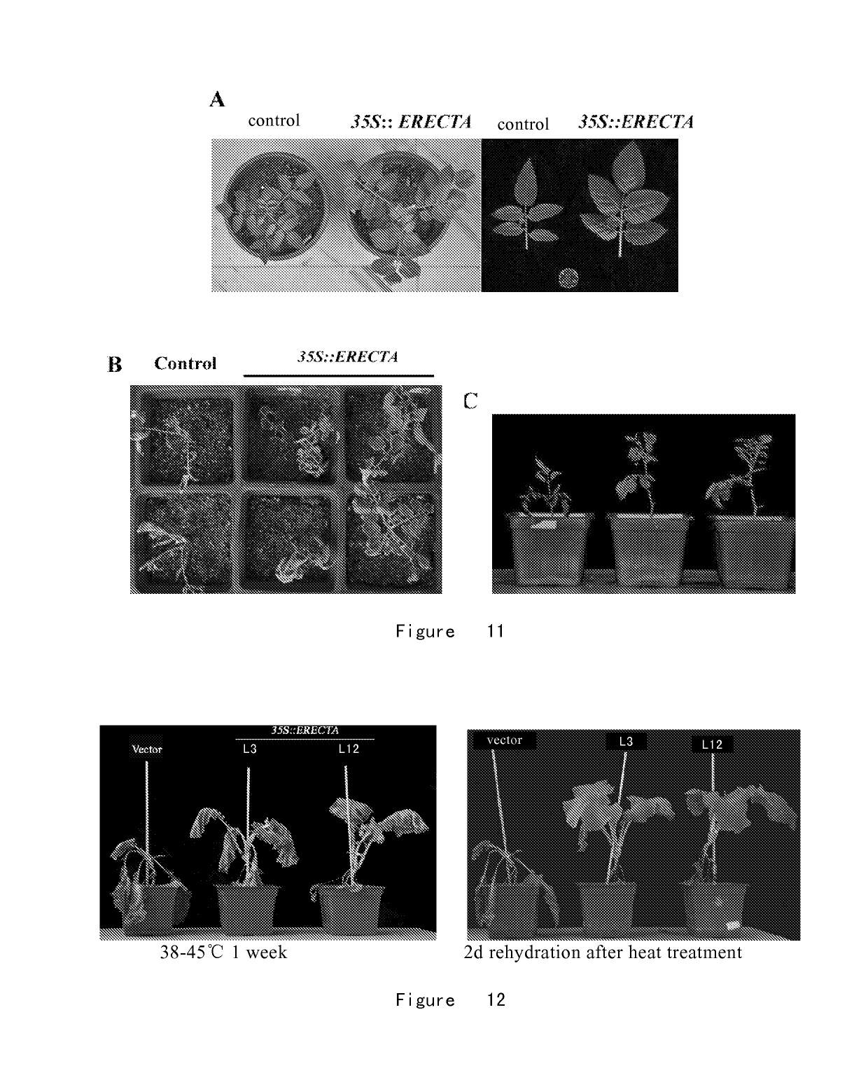

The present inventor transferred ERECTA gene promoted by 35S (p35S::ERECTA) to Arabidopsis Col-0 and obtained 9 ERECTA-overexpressing transgenic lines. Realtime PCR was used to detect the amount of ERECTA gene expression in these transgenic lines, wherein the amount of ERECTA gene expression of line L2-3 was raised about 40 times compared with the wild-type, while L7-1 raised about 80-fold (FIG. 1A). So these two lines were selected to perform the following morphological analysis and follow-up experiments.

At the seedling stage (growing in ½ MS medium for 10 days), overexpression of ERECTA led to increase in cotyledon and euphylla, petioles also increased (FIG. 1B). When plants moved to soil grew 20 days to reach rosette leaf stage, it can be found that all rosette leaves have a certain degree of increase, and this increase was positively correlated with the amount of ERECTA gene expression (FIG. 1C).

### Example 3

### Overexpression of ERECTA Affected Leaf Development by Promoting Cell Elongation

To further observe the morphological characteristics of overexpressing plant, the present inventor used the ninth sheet of rosette leaves as an example (FIG. 2A), and analyzed cytology structure of the blade of ERECTA-overexpressing plant.

The present inventor measured the width and length of the blade, respectively, and statistical analysis of the data indicated that the width, length of the blade of overexpressing lines significantly increased compared with the wild-type (FIG. 2C, D), further anatomical analysis of cross-section of the blade showed that leaf epidermal cells and mesophyll cells of overexpressing lines were larger compared with the wild type. Therefore, the cell enlargement led to the larger blade (FIG. 2B).

### Example 4

### ERECTA Overexpression Promoted the Development of Plant Side Moss

Further, the morphological features of ERECTA-overexpressing plants after bolting were observed. The inventor found that, the height of overexpressing line showed no significant change compared with the wild-type (FIG. 3A), but the number of side moss and branch number of the main moss both significantly increased, leading to significant increase in the number of inflorescences (FIG. 3A, B), in turn resulting in increased biomass (FIG. 3A).

### Example 5

### ERECTA Overexpression LED to Reduced Stomatal Density and Increased Transpiration Rate

The present inventor used scanning electron microscopy to detect stomatal development changes of overexpressing lines. The results of electron microscope were shown in FIG. 4, compared with the wild-type Col-0, the stomatal density of loss-of-function mutant of ERECTA, er-105 increased about 2-fold (FIG. 4A, B), but stomatal coefficient didn't change (FIG. 4C).

In contrast, stomatal density became significantly smaller in ERECTA overexpressing line. The stomatal density of 35S::ERECTA L2-3 decreased by 36%, L7-1 decreased by 55% as compared with the wild-type (FIG. 4 A, B). But as for stomatal coefficient, like mutant, overexpressing plants did not change significantly compared with the wild-type (WT) (FIG. 4C).

The decrease in stomatal density is often accompanied by the change of transpiration efficiency. Therefore, the present inventor measured transpiration efficiency of ERECTA overexpressing line. As shown in FIG. 10, transpiration efficiency of Arabidopsis L7-1 line was significantly higher than the wild-type. This proved that ERECTA overexpression can improve instantaneous water use efficiency of plant.

The results proved that ERECTA was a negative regulation factor of stomatal differentiation, but didn't affect stomatal coefficient. At the same time, ERECTA can regulate transpiration efficiency (instantaneous water use efficiency) by affecting stomatal differentiation.

### Example 6

### Identification of ERECTA-Overexpressing Plant Resistance to High Temperature Stress

To further verify the role of ERECTA gene in plant resistance to high temperature stress, the present inventor performed high temperature stress treatment on the obtained ERECTA-overexpressing materials. When treated under 40° C. for 48 h, the whole seedling of er-105 became dark green and then withered, nearly died (FIG. 5A). At this time some leaves of the wild-type Col-0 were dark green and wilting, petiole and new leaves presented a normal bright green state (FIG. 5A). While the status of the two overexpressing lines was better than that of the wild-type: for L2-3, the old leaves and mature leaves and the edges of new leaves were wilting and withering, the majority tissues showed a bright green state. The resistance of L7-1 was stronger compared with L2-3, there was virtually no wilting necrotic areas on the leaves, only local injury was observed (FIG. 5A).

After rehydration at room temperature for 2-3 days following high temperature treatment for 48 hours, the statistical survival rate was consistent with the observed state before rehydration, survival rate of the two ERECTA-overexpressing lines were both higher than that of the wild-type: survival rate of Col-0 was about 48%, survival rate of er-105 was less than 20%, survival rate of L2-3 increased to 65%, increased by 30% as compared with the wild-type; survival rate of L7-1 was about 75%, increased by 50% as compared with the wild-type (FIG. 5B).

The above results demonstrated that ERECTA was indeed involved in the high temperature tolerance of the plant, increase amount of its expression significantly enhanced high temperature resistance.

### Example 7

### ERECTA Mediated High Temperature-Induced Cell Death

The present inventor also measured conductivity of the wild type, er mutant and overexpressing plants under high temperature stress. Conductivity was also known as ion permeability. When cells were injured, cell membrane permeability increased and ion exosmosis started, cell death occurred when the membrane was damaged to a certain extent. Conductivity therefore reflected the parameters of cell membrane integrity, was physiological index for characterization of cell death. The larger the value reflected the more severe the degree of cell death.

At 0 h, 12 h, 24 h, 36 h and 48 h of high temperature stress, conductivity measurement results were shown in FIG. 6. It can be seen that, with respect to the wild-type or er mutant, the level of ion leakage in ERECTA transgenic plants reduced. Therefore, ERECTA alleviated high temperature-induced cell death.

### Example 8

### ERECTA-Overexpressing Plant Had Resistance to 30° C. High Temperature Stress

Normal growth temperature of Arabidopsis was 21° C. to 23° C., the temperature of 40° C. was extreme high temperature to Arabidopsis. To further identify whether ERECTA is involved in high temperature stress, the present inventor detected survival rate of overexpressing line at 30° C.

The results were shown in FIG. 7A, the leaves of the plant were small and the petioles were slender growing at 30° C. After 30° C. treatment for 30 days, the leaves of the wild-type gradually turned yellow, the whole plant wilted and showed a death state. While for ERECTA-overexpressing plant line L7-1, most of the leaves remained green. After rehydration for 2-3 days, statistical survival rate of wild-type was less than 15%, and that of L7-1 was more than 40% (FIG. 7B).

At the same time, for ERECTA transgenic tomato and oilseed rape, also observed significant enhancement of heat resistance.

Thus, ERECTA can enhance the resistance of plants to long-term high temperature stress.

### Example 9

### Screening or Breeding Methods

The starting plants: wild-type Arabidopsis and Col-0. Performed gene transfer operation on this plant species, transferred ERECTA gene to identify whether its heat resistance can be improved. Produced transgenic plants as the aforementioned method, obtained 1#, 2# transgenic plants.

By conventional Western blotting or Realtime PCR method, detected ERECTA expression in #1, #2 transgenic plants; its expression level was higher than that of the starting variety Col-0 by 50% or more, so 1#, 2# transgenic plants can be determined as potential plants having heat resistance.

### Example 10

### ERECTA Variants

The present inventor analyzed ERECTA protein domain, found that its carboxy-terminal part was the main region to perform the function. The amino-terminal region was not important region of functioning (LRR domain); changes may be made on many sites of the amino-terminal region.

Use a coding sequence to replace the sequence in ClaI/BamHI of p35S::ERECTA, the protein encoded by said coding sequence had sequence similar to SEQ ID NO: 3, different only in that position 33 was Leu (for the wild-type protein, Ile). Ile and Leu both belonged to aliphatic neutral amino acids and had similar structure; this locus mutation had little effect on the activity of the protein.

Use a coding sequence to replace the sequence in ClaI/BamHI of p35S::ERECTA, the protein encoded by said coding sequence had sequence similar to SEQ ID NO: 3, different only in that position 386 was Ala (for the wild-type protein, Val). Val and Ala both belonged to aliphatic neutral amino acids and had similar structure; this locus mutation had little effect on the activity of the protein.

Use a coding sequence to replace the sequence in ClaI/BamHI of p35S::ERECTA, the protein encoded by said coding sequence had sequence similar to SEQ ID NO: 3, different only in that the position 213 was Pro (for the wild-type protein, Gly). Pro and Gly were similar amino acids; this locus mutation had little effect on the activity of the protein.

Use a coding sequence to replace the sequence in ClaI/BamHI of p35S::ERECTA, the protein encoded by said coding sequence had sequence similar to SEQ ID NO: 3, different only in that amino acid Gly was inserted into the intermediate section between position 278 and 279. Gly was the smallest amino acid and was a neutral amino acid, inserting in said sites had substantially no effect on the three-dimensional structure of the protein, and didn't affect the activity of the protein.

Use a coding sequence to replace the sequence in ClaI/BamHI of p35S::ERECTA, the protein encoded by said coding sequence had sequence similar to SEQ ID NO: 3, different only in that position 502 lacked Ile. Said locus deletion had no effect on the three-dimensional structure of the protein, didn't affect the activity of the protein.

Expression plasmid obtained above was prepared as the preceding method to produce transgenic Arabidopsis thaliana (Agrobacterium method), identified heat resistance ability of obtained plants, i.e., as the preceding method, heat resistance was measured under 30° C. high temperature stress. The results showed that, after 30° C. treatment for 30 days, most leaves of these plants remained green.

### Example 11

### ERECTA Overexpression Improved Heat Resistance of Tomato

35S promoter-driven ERECTA-overexpressing vector (p35S::ERECTA) was transferred to a high-temperature-sensitive tomato variety LA1589, and obtained more than 20 independent T₀ generation transgenic lines containing 35S::ERECTA. Empty vector transgenic plants were used as control. Similar to transgenic Arabidopsis plant, ERECTA overexpression in tomato also led to significant increase in the leaves, as shown in FIG. 11A.

For T₀ generation plants (including no-load control), took the same size top for cuttings, cultured at 25° C. to get the seedlings growing at the same level. After two weeks, the seedlings were placed in a 30° C. incubator and performed high temperature treatment. Treatment temperature gradually increased to 38° C. in 4 days. The final treatment temperature reached 43-45° C. to treat for three days. The control plants (no load) were completely dead, but the transgenic plants were still alive (FIG. 11B); After rehydration, control plants can not be restored, and the transgenic plants can restore growth to some extent, showing resistance to extreme high temperature, as shown in FIG. 11C. This demonstrated that in high temperature sensitive crops, ERECTA overexpression can improve its heat resistance.

### Example 12

### ERECTA Overexpression Increased Heat Resistance of Rape

The present inventor transferred 35S promoter-driven ERECTA-overexpressing vector (p35S::ERECTA) to a rape line “Zheshuang 758” and obtained more than 15 independent T₀ generation transgenic lines. The present inventor directly put the 30-day-old transgenic seedlings to high temperature treatment; empty vector transgenic seedlings were used as control. Within 4 days, the temperature gradually increased from 30° C. to 38° C., after which initiated the extreme high temperature treatment of 43-45° C. When high temperature treatment proceeded to 48 hours, severe wilting can be observed in the leaves of no-load control transgenic lines; while the leaves of ERECTA transgenic plants (L3, L12) also showed a certain degree of wilting, but still maintained a certain degree of turgor. After rehydration, control plants could not survive, and ERECTA transgenic plants could restore growth to a large extent, as shown in FIG. 12.

The above data of transgenic tomato and transgenic rape demonstrated that in high temperature sensitive crops, ERECTA overexpression can improve its heat resistance.

All references cited in the present invention are incorporated herein by reference as each one of them was individually cited. Further, it should be understood that various modifications and/or changes are obvious to a skilled person in the art, in view of above teaching of the subject invention, which all fall within the scope defined by the appended claims.

1. Method of improving heat-resistant ability of plains; promoting plant development; increasing plant yield; increasing plant biomass; reducing stomatal density of plants; or improving water use efficiency of plants which comprises applying ERECTA protein or the polynucleotide encoding therefor to a plant, plant cell, plant tissue, organ or seed.

2. A method of preparing a plant with improved heat-resistant ability, rapid development, increased yield, increased biomass, low stomatal density or high water use efficiency which comprises applying ERECTA protein or the polynucleotide encoding therefor to a plant, plant cell, plant tissue, organ or seed.

3. The method according to claim 1 or 2, wherein said ERECTA protein is:
(a) a protein with amino acid sequence as set forth in SEQ ID NO:3; or
(b) a protein derived from (a) by substitution, deletion or addition of one or more residues in the amino acid sequence of SEQ ID NO:3 and having the ability to improve plant heat resistance: or
(c) a polypeptide, having more than 70% identity to the amino acid sequence defined in (a) and having the ability to improve plant heat resistance; or
(d) a protein fragment of SEQ ID NO:3 and having the function of (a) protein.

4. The method according to claim 1 or 2, wherein, the polynucleotide encoding ERECTA protein is:
(i) a polynucleotide having a sequence as set forth in SEQ ID NO:1;
(ii) a polynucleotide, the nucleotide sequence of it can hybridize with polynucleotide sequence defined in (i) under stringent conditions and encoding a protein having the function to improve plant heat resistance;
(iii) a polynucleotide, the nucleotide sequence of it has more than 70% identity with nucleotide sequence defined in (i) and encoding a protein having the function to improve plant heat resistance; or
(iv) a polynucleotide, having sequence complimentary to the sequence as set forth in SEQ ID NO:1.

5. The method according to claim 1 or 2, wherein, the polynucleotide encoding ERECTA protein is:
(i′) a polynucleotide having a sequence as set forth in SEQ ID NO:2:
(ii′) a polynucleotide, the nucleotide sequence of it can hybridize with polynucleotide sequence defined in (i′) under stringent conditions and encoding a protein having the function to improve plant heat resistance;
(iii′) a polynucleotide, the nucleotide sequence of it has more than 70% identity with nucleotide sequence defined in (i′) and encoding a protein having the function to improve plant heat resistance; or
(iv′) a polynucleotide, having sequence complementary to the sequence as set forth in SEQ ID NO:2.

6. The method according to claim 1 or 2, wherein, said plant is selected from the group consisting of, but not limited to: Cruciferae, Gramineae or Solanaceae.

7. The method according to claim 6, wherein, said plant is selected from the group consisting of, but not limited to: oilseed rape, Chinese cabbage, Little cabbage, beet, rice, wheat, barley, maize, rye, sorghum, soybean, tomato, pepper, potato, tobacco, wolfberry.

8. A method for improving heat-resistant ability of plants, promoting plant development, improving plant yield, increasing plant biomass, reducing stomatal density or improving water use efficiency of plants, said method comprises: improving the expression or activity of ERECTA protein in plants.

9. A method for producing a plant with improved heat-resistant ability, rapid development, increased yield, increased biomass, low stomatal density or high water use efficiency, said method comprises: improving the expression or activity of ERECTA protein in plants.

10. The method according to claim 8 or 9, wherein, said method comprises: transferring the polynucleotide encoding ERECTA protein to the plant.

11. The method according to claim 10, wherein, said method comprises the steps of:
(i) providing agrobacterium strain containing an expression vector containing a polynucleotide encoding ERECTA protein:
(ii) contacting a plant cell, tissue or organ with the agrobacterium strain of step (i), so that said polynucleotide encoding ERECTA protein is transferred to the plant.

12. The transgenic plants or their hybrids according to claim 8 or 9, wherein said transgenic plants or hybrids have improved heat-resistant ability, rapid development, increased yield, increased biomass, low stomatal density er high water use efficiency, compared with control plants.

13. Seeds obtained from a transgenic plants prepared according to claim 8 or 9.

14. A method to identify heat-resistant ability, development conditions, production, biomass, stomatal density or water use efficiency of plants which comprises introducing ERECTA protein or the polynucleotide encoding therefor and utilizing said protein or polynucleotide as a molecular market for such properties.

15. A method for identifying heat-resistant ability, development conditions, production, biomass, or stomatal density of plants, the method comprises: detecting ERECTA protests expression in plant to be tested; if the expression of the polypeptide in plant to be tested is higher than normal value of ERECTA protein expression in the plant, said plant is the plant having heat-resistant ability, good development, high-yield, high biomass or low stomatal density; if the expression of the polypeptide in plant to be tested is less than normal value of ERECTA protein expression in the plant, said plant is the plant not having heat-resistant ability and having low-yield, low biomass or high stomatal density.
